(19) United States
(12) Patent Application Publication
(10) Pub. No.: US 2019/0284546 A1
(43) Pub. Date: Sep. 19, 2019

(54) METHOD FOR CONTROLLING KINETICS OF A CULTURED CELL

(71) Applicants: National University Corporation Kumamoto University, Kumamoto (JP); Ushio Denki Kabushiki Kaisha, Tokyo (JP); Kyushu University, National University Corporation, Fukuoka (JP)

(72) Inventors: Yuta NAKASHIMA, Kumamoto-shi (JP); Kinichi MORITA, Tokyo (JP); Kosuke TASHIRO, Fukuoka-shi (JP)

(73) Assignees: National University Corporation Kumamoto University, Kumamoto (JP); Ushio Denki Kabushiki Kaisha, Tokyo (JP); Kyushu University, National University Corporation, Fukuoka (JP)

(21) Appl. No.: 16/348,863

(22) PCT Filed: Nov. 14, 2017
(86) PCT No.: PCT/JP2017/040898

§ 371 Date: May 9, 2019

(30) Foreign Application Priority Data
Nov. 15, 2016 (JP) 2016-222215

(51) Int. Cl. C12N 13/00; A61L 27/50; A61L 27/06; A61L 27/38

(52) U.S. Cl. CPC C12N 13/00; A61L 27/50; A61L 2400/18; A61L 27/38; A61L 2300/64; A61L 27/06

(57) ABSTRACT

A method for controlling the kinetics of a cultured cell includes: a culture step of culturing a cell adhered to the surface of a base material including titanium oxide having an anatase structure on the surface; an irradiation step of irradiating the surface with light in a wavelength range in which the titanium oxide exhibits photocatalytic activity during culture of the cell; and a control step of controlling the kinetics of the cell by controlling the irradiation amount of the light to the surface. The wavelength range may be 400 nm or less.

### TECHNICAL FIELD

The present disclosure relates to a method for controlling the kinetics of a cultured cell.

### BACKGROUND ART

Cells are cultured for the purpose of clarifying properties or functions of the cells, development and production of medicines, or the like. Also in regenerative medicine, which has been studied in recent years, stem cells and the like are cultured before transplantation into patients. Since cultured cells are important in various applications, techniques for controlling the kinetics of cultured cells are demanded.

The kinetics of a cultured cell is influenced by the culture temperature, the composition of a culture medium, a base material used for culturing the cell, and the like. As the base material for culturing a cell, titanium oxide is employed from the viewpoint of cell adhesion and the like. For example, a cell adhesive base material having a titanium oxide film is disclosed in Patent Literature 1. A cell culture vessel including a resin layer containing a titanium oxide photocatalyst on a cell culture surface is disclosed in Patent Literature 2. It is described that, in the cell culture vessel, a cultured cell can be easily detached from the cell culture surface by utilizing the property of a titanium oxide photocatalyst to hydrophilize by light irradiation.

Titanium oxide is used not only for cell culture but also for biological implants and the like. For example, a biological implant whose surface is coated with titanium oxide is disclosed in Patent Literature 3. By irradiating the biological implant with ultraviolet light, an organic compound on the surface of the biological implant can be decomposed and removed by photocatalysis.

### CITATION LIST

### Patent Literature

Patent Literature 1: Unexamined Japanese Patent Application Kokai Publication No. 2002-253204

Patent Literature 2: Unexamined Japanese Patent Application Kokai Publication No. 2004-51

Patent Literature 3: Unexamined Japanese Patent Application Kokai Publication No. 2014-193249

### SUMMARY OF INVENTION

### Technical Problem

The above-described cell adhesive base material disclosed in Patent Literature 1 above and the cell culture vessel disclosed in Patent Literature 2 utilize titanium oxide to improve the convenience in cell culture, such as securing cell adhesion and easy detachment of cells. The biological implant disclosed in Patent Literature 3 finds utility of titanium oxide for cleaning the surface of the biological implant. However, in any of Patent Literature 1 to 3, the influence of titanium oxide on the kinetics of a cultured cell has not been studied at all.

Flexible control of the kinetics of cultured cells by utilizing titanium oxide is useful not only to make cell culture convenience but also to stably provide cultured cells for various uses such as biological experiments, evaluation of medicines, and production of materials for antibody medicine and regenerative medicine.

The present disclosure has been made in view of the above-described circumstances, and an objective of the present disclosure is to provide a method for controlling the kinetics of a cultured cell in which the kinetics of the cultured cell is flexibly controlled.

### Solution to Problem

The present inventors focused on the photocatalytic activity of titanium oxide, and intensively studied an effect of the photocatalytic activity on the kinetics of a cultured cell to complete the present disclosure.

The method for controlling the kinetics of a cultured cell according to an aspect of the present disclosure includes:

a culture step of culturing a cell adhered to the surface of a base material including titanium oxide having an anatase structure on the surface;

an irradiation step of irradiating the surface with light in a wavelength range in which the titanium oxide exhibits photocatalytic activity during culture of the cell; and

a control step of controlling the kinetics of the cell by controlling an irradiation amount of the light to the surface.

In this case, preferably, the wavelength range is

400 nm or less.

Preferably, in the irradiation step,

the light is irradiated during lag phase, logarithmic growth phase, or stationary phase in cell culture.

Preferably, the titanium oxide is

titanium oxide obtained by irradiating titanium with vacuum ultraviolet light.

### Advantageous Effects of Invention

According to the present disclosure, the kinetics of a cultured cell can be flexibly controlled.

### BRIEF DESCRIPTION OF DRAWINGS

FIG. 1 is a diagram showing a sectional view of one aspect of the use of Example 1;

FIG. 2 is a diagram showing the absorbance of a mouse-derived osteoblast sample measured with a spectrophotometer;

FIG. 3 is a diagram showing the absorbance of a mouse-derived osteoblast sample measured with an absorption spectrometer;

FIG. 4 is a diagram showing the absorbance of a mouse-derived myoblast sample measured with a spectrophotometer; and

FIG. 5 is a diagram showing the absorbance of a mouse-derived myoblast sample measured with an absorption spectrometer.

### DESCRIPTION OF EMBODIMENTS

An embodiment according to the present disclosure will be described with reference to the attached drawings. The present disclosure is not limited by the following embodiment and drawings.

### Embodiment

A method for controlling the kinetics of a cultured cell according to the present embodiment includes a culture step, an irradiation step, and a control step. First, the culture step will be described. In the culture step, a cell adhered to the surface of a base material provided with titanium oxide having an anatase structure on the surface is cultured. Preferably, the titanium oxide having an anatase structure is titanium oxide obtained by irradiating titanium with vacuum ultraviolet (Vacuum Ultra Violet, hereinafter also simply referred to as “VUV”).

Hereinafter, oxidation of titanium by VUV will be described in detail, taking as an example a case in which a titanium oxide layer is formed on the surface of a base material made of titanium. For example, when a base material made of titanium is irradiated with VUV, the surface of the base material irradiated with VUV is oxidized. The wavelength of VUV is preferably from 10 to 200 nm, more preferably from 50 to 180 nm, and still more preferably from 100 to 175 nm. The illuminance of VUV on the base material is not particularly limited, and is, for example, from 1 to 100 mW/cm², from 3 to 50 mW/cm², or from 5 to 20 mW/cm². The irradiation time of VUV is not particularly limited, and is, for example, from 3 to 60 minutes, from 5 to 40 minutes, or from 10 to 30 minutes in the case of the above-described illuminance

When VUV is irradiated in an atmosphere containing oxygen, the oxygen in the atmosphere is decomposed into atomic oxygen as shown by the following formula.

O₂+hv→O(³P)+O(³P)

Here, O (³P) represents atomic oxygen in the ground state. The atomic oxygen in the ground state generates ozone in a three-body collision reaction between oxygen in the atmosphere and a third body M of the reaction, as in the following formula. M removes excess energy generated by the reaction, and stabilizes ozone.

O(³P)+O₂+M→O₃+M

Further, ozone is decomposed by VUV, and generates excited singlet oxygen (O (¹D)) as in the following formula.

O₃+hv→O(¹D)+O₂

It is considered that the surface of the base material is oxidized by the generated excited singlet oxygen, and a titanium oxide (TiO₂) layer is formed on the surface.

In addition, when the wavelength of VUV is 175 nm or less, the above-described excited singlet oxygen is also generated by direct decomposition of an oxygen molecule as in the following formula.

O₂+hv→O(¹D)+O(¹D)

When moisture is attached to the surface of the base material, a reaction between VUV and water produces a hydroxide ion (OH⁻) as in the following formula.

H₂O+hv→H⁺+OH⁻

Titanium on the surface of the base material is also oxidized by a hydroxide ion. Oxidation of titanium on the surface of the base material increases the hydrophilicity of the surface of the base material.

It is preferable that all of the titanium oxide on the surface of the base material according to the present embodiment have an anatase structure, and at least a part of the titanium oxide may have an anatase structure. As described above, when titanium on the surface of the base material is oxidized by irradiating VUV, it is considered that titanium oxide having an anatase structure and titanium oxide having a rutile structure are mixed on the surface of the base material. It is possible to confirm that the titanium oxide on the surface of the base material has an anatase structure by analyzing the surface by, for example, Raman spectroscopy.

In the culture step, for example, by immersing the above-described base material in a culture dish holding a culture medium, and plating a cell on the surface of the base material, the cell plated on the surface of the base material adheres to the surface of the base material directly or through extracellular matrix. The cell is not particularly limited, and examples thereof include an animal cell, a plant cell, an insect cell, a stem cell, an iPS cell, and a bacterium. The cell may be a primary cell or a passaged cell line.

The cell is cultured by a known method. Culture conditions such as the composition of the culture medium and the culture temperature are appropriately set according to a cell to be cultured. For example, the cell can be cultured by leaving the above-described culture dish in an incubator for a predetermined time. The culture time may be any time depending on the cell.

Next, the irradiation step will be described. In the irradiation step, the surface is irradiated with light in a wavelength range in which titanium oxide exhibits photocatalytic activity during cell culture. The wavelength range in which titanium oxide exhibits photocatalytic activity is, for example, 400 nm or less or 378.5 nm or less, preferably from 200 to 400 nm, from 250 to 400 nm, from 300 to 400 nm, or from 350 to 400 nm.

Light irradiation in the irradiation step may be continued during the culture or may be limited to a part of the culture time. For example, in the irradiation step, light may be irradiated during lag phase, logarithmic growth phase, or a stationary phase in cell culture. In this case, the light irradiation may be performed, for example, between the lag phase and the logarithmic growth phase, or may be performed in the lag phase and the stationary phase.

Subsequently, the control step will be described. In the control step, the kinetics of a cell is controlled by controlling the irradiation amount of light applied to the surface. The irradiation amount of light can be controlled, for example, by controlling at least one of the irradiation time of light and the illuminance on the base material. The irradiation time of light and the illuminance of light may be appropriately set according to the kinetics desired to be given to the cell.

Here, the influence of photocatalytic activity of titanium oxide on the cultured cell is described. In titanium oxide having an anatase structure, the energy difference Eg (band gap) between the upper end of the valence band and the lower end of the conduction band is 3.2 eV. When light of a wavelength range in which titanium oxide exhibits photocatalytic activity, for example, ultraviolet light (Ultra Violet, hereinafter also simply referred to as “UV”) is irradiated to titanium oxide having an anatase structure, an electron in the valence band is transferred to the conductor, and a hole is generated in the conductor. As a result, the surface of titanium oxide is in a state in which photocatalytic reaction is possible.

A hole generated in UV-irradiated titanium oxide oxidizes and decomposes water in the culture medium, and a hydroxide ion (OH⁻) is generated. The hydroxide ion reduces sodium ion (Nat) excreted out of a cell by, for example, a membrane protein (Na⁺-ATPase) of a cell membrane which drives a sodium-potassium pump. For example, a membrane protein (H⁺-ATPase) of a cell membrane that drives a proton pump reduces a hydrogen ion (H⁺) excreted out of the cell. For this reason, the membrane potential, which is the potential difference between the extracellular and cytoplasmic, decreases.

For example, a sodium-potassium pump drains three sodium ions out of the cell, and takes up two potassium ions (K⁺) in the cytoplasm. As the membrane potential decreases due to this ion concentration difference, the amount of current flowing into the cell also decreases. As a result, synthesis of adenosine triphosphate in the cell is suppressed.

As described above, it is considered that, when UV is irradiated to a base material including titanium oxide having an anatase structure on the surface, the membrane potential of the cell adhering to the surface decreases by photocatalytic activity. For this reason, in the control step, the kinetics of a cell can be changed according to the irradiation amount of light. The reduction of the membrane potential due to the photocatalytic activity is an example of the influence of the photocatalytic activity of titanium oxide, and is not limited thereto.

By the method for controlling the kinetics of a cultured cell according to the present embodiment, it is possible to flexibly change the cell cycle, increase/decrease in the number of cells in a predetermined time, the proliferation curve, the energy metabolic efficiency, the timing of differentiation and/or the like. For example, in the control step, when the irradiation amount of light is controlled to an irradiation amount determined by a preliminary experiment or the like, it is possible to control the kinetics of a cultured cell, such as to induce a desired kinetics of the cultured cell.

### EXAMPLES

The present disclosure will be more specifically described by the following Examples, but the present disclosure is not limited by the Examples.

Preparation of Base Material with Titanium Oxide on Surface

A titanium base material (width 20 mm, depth 20 mm, and thickness 1 mm) installed on a support was irradiated with VUV having a wavelength of 172 nm emitted from a VUV lamp from above for 15 minutes to oxidize titanium contained on the surface and in the vicinity of the surface of the titanium base material to prepare a titanium base material including titanium oxide on the surface (Example 1). As the VUV lamp, a Xe excimer lamp (Min-Excimer, SUS713, manufactured by USHIO INC.) was used. The illuminance of 172 nm wavelength light on the surface of the titanium base material was 10 mW/cm². In Comparative Example, a titanium base material (width 20 mm, depth 20 mm, and thickness 1 mm) not irradiated with VUV was also used.

Cell Culture

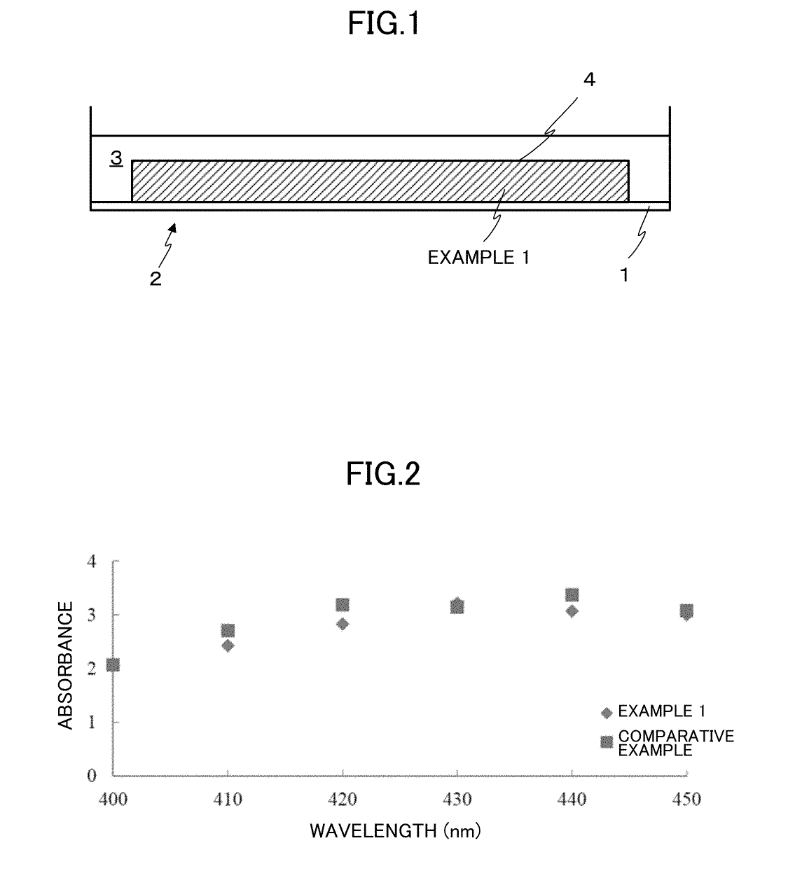

As shown in FIG. 1, Example 1 was immersed in a culture medium 3 in a culture petri dish 2 in which the surface of the bottom was coated with a fluorine coating 1, and a cell was plated on a surface 4 of Example 1, and the cell was cultured. The culture petri dish 2 was allowed to stand still in an incubator (APC-30D, manufactured by ASTEC CO., Ltd.) and cultured at 37° C. in a 5% CO₂ environment for 67 hours. During the culture, room light was applied to Example 1 in the culture petri dish 2. A cell was similarly cultured in Comparative Example

The cells used were a mouse-derived osteoblast MC3T3-E1 and a mouse-derived myoblast C2C12. The culture medium for MC3T3-E1 was 5 ml of liquid medium MEMα (137-17215, manufactured by Wako Pure Chemical Industries Ltd.). The seeding density of MC3T3-E1 was 1.0×10⁵ cells/ml. On the other hand, the culture medium for C2C12 was 5 ml of liquid medium D-MEM (043-30085, manufactured by Wako Pure Chemical Industries Ltd.). The seeding density of C2C12 was 1.0×10⁵ cells/ml.

Evaluation of Cultured Cells

The cultured cells were evaluated by colorimetric assay using a tetrazolium salt (WST-1) as follows. As evaluation reagents, Reagent A (WST-1/HEPES) and Reagent B (1-Methoxy PMS) of Cell Counting Kit (manufactured by Dojindo Molecular Technologies, Inc.) were used. First, 500 μl of a mixed solution of Reagent A and Reagent B was added to the culture petri dish 2 after culture. Next, the culture petri dish 2 was kept in an incubator for 1 hour. By this, WST-1 is converted to formazan dye by mitochondrial dehydrogenase in a living cell.

Then, 4 ml of the culture solution was collected from the culture petri dish 2 removed from the incubator, and poured into a cuvette, and the absorbance was measured. A spectrophotometer U-5100 (manufactured by Hitachi, Ltd.) was used for measurement of absorbance. The measurement wavelengths were 400 nm, 410 nm, 420 nm, 430 nm, 440 nm, and 450 nm.

After measuring the absorbance, a sample partially collected from the cuvette was injected into a PCR tube for absorption spectrometer. The absorbance at from 400 to 540 nm was measured using an absorption spectrometer PAS-110 (manufactured by USHIO INC.).

Results

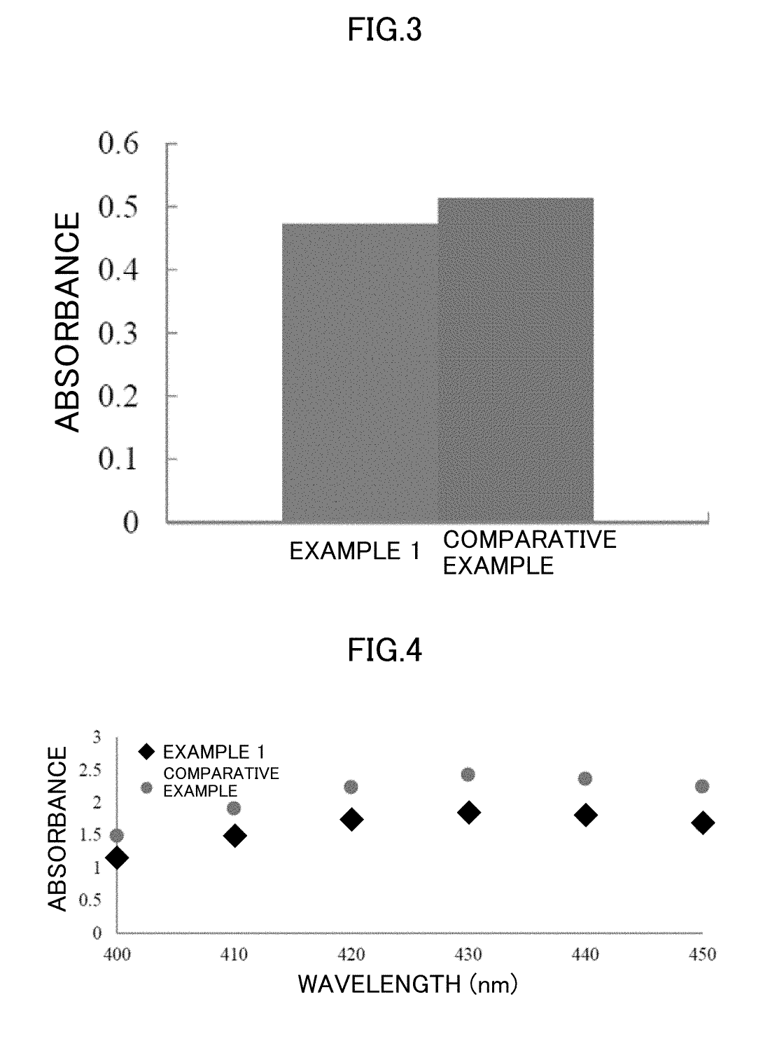

The absorbance measured with a spectrophotometer and the absorbance measured with an absorption spectrometer for MC3T3-E1 are shown in FIG. 2 and FIG. 3, respectively. According to FIG. 2, the absorbances at 400 nm and 430 nm of Example were comparable to those of Comparative Example, and the absorbances at 410 nm, 420 nm, 440 nm, and 450 nm of Example 1 are lower than those of Comparative Example. As shown in FIG. 3, the absorbance at from 400 to 540 nm of Example 1 was lower than that of Comparative Example

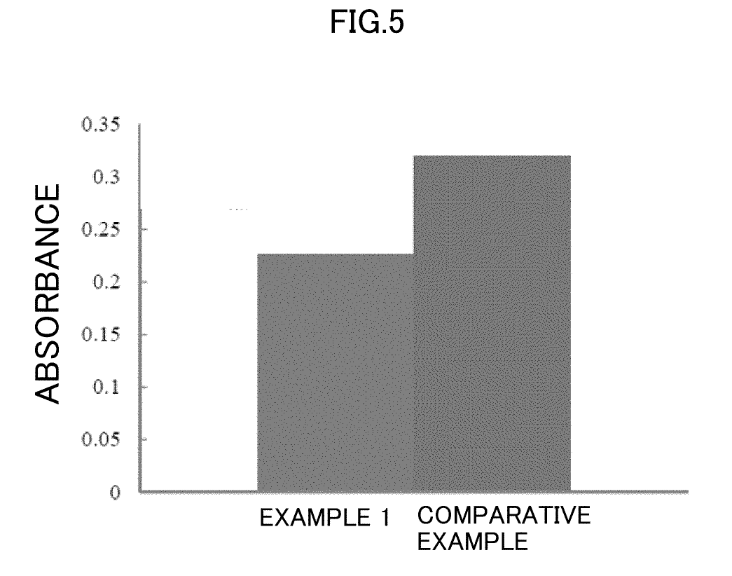

Similarly, for C2C12, the absorbance measured with a spectrophotometer and the absorbance measured with an absorption spectrometer are shown in FIG. 4 and FIG. 5, respectively. As shown in FIG. 4, the absorbance measured with a spectrophotometer was lower at all wavelengths in Example than in Comparative Example. As shown in FIG. 5, the absorbance at from 400 to 540 nm of Example 1 was lower than that of Comparative Example

From the above results, it was shown that the amount of mitochondrial dehydrogenase in the cell adhered and cultured in Example 1 was smaller than the amount of mitochondrial dehydrogenase in the cell adhered and cultured in Comparative Example Therefore, it is considered that the kinetics of the cell adhered and cultured in Example 1 is different from that of the cell adhered and cultured in Comparative Example. This is considered to be because a photocatalytic reaction was caused by a light in a wavelength range in which titanium oxide exhibited photocatalytic activity contained in the room light irradiated to Example 1 and Comparative Example during cell culture, and the membrane potential of the cell adhered and cultured in Example 1 decreased.

As shown in Example, when culturing a cell on the surface of a titanium oxide layer of a base material including the titanium oxide layer having an anatase structure and having photocatalytic activity on the surface, by irradiating the surface of the titanium oxide layer with light during cell culture and causing a photocatalytic reaction, it is possible to change the kinetics of the cultured cell. In other words, during cell culture on the base material, by controlling the irradiation time or illuminance of light in a wavelength range in which titanium oxide exhibits photocatalytic activity on the surface of the titanium oxide layer, it is possible to control the kinetics of the cultured cell.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

This application claims the benefit of Japanese Patent Application No. 2016-222215, filed on Nov. 15, 2016, the entire disclosure of which is incorporated by reference herein.

### REFERENCE SIGNS LIST

1 Fluorine coating
2 Culture petri dish
3 Culture medium

### 4 Surface

1. A method for controlling kinetics of a cultured cell, the method comprising:
a culture step of culturing a cell adhered to a surface of a base material including titanium oxide having an anatase structure on the surface;
an irradiation step of irradiating the surface with light in a wavelength range in which the titanium oxide exhibits photocatalytic activity during culture of the cell; and
a control step of controlling the kinetics of the cell by controlling an irradiation amount of the light to the surface.

2. The method according to claim 1, wherein the wavelength range is 400 nm or less.

3. The method according to claim 1, wherein in the irradiation step, the light is irradiated during lag phase, logarithmic growth phase, or stationary phase in cell culture.

4. The method according to claim 1, wherein the titanium oxide is titanium oxide obtained by irradiating titanium with vacuum ultraviolet light.
